(19) United States
(12) Patent Application Publication
(10) Pub. No.: US 2019/0111436 A1
(43) Pub. Date: Apr. 18, 2019

(54) SINGLE-SIDED HEAT TRANSFER INTERFACE FOR A DIAGNOSTIC ASSAY SYSTEM

(71) Applicant: Integrated Nano-Technologies, Inc., Henrietta, NY (US)

(72) Inventors: Nathaniel E. Wescott, West Henrietta, NY (US); Dennis M. Connolly, Rochester, NY (US); Richard S. Murante, Rochester, NY (US); Mark J. Smith, Rochester, NY (US)

(73) Assignee: Integrated Nano-Technologies, Inc., Henrietta, NY (US)

(21) Appl. No.: 16/206,253

(22) Filed: Nov. 30, 2018

Related U.S. Application Data
(63) Continuation-in-part of application No. 16/303,441.
(60) Provisional application No. 62/344,711, filed on Jun. 2, 2016.

(51) Int. Cl. B01L 9/00; B01L 7/00; C12Q 1/686

(52) U.S. Cl. CPC B01L 9/00; B01L 7/52; B01L 2300/1861; B01L 2200/04; B01L 2300/1827; C12Q 1/686

(57) ABSTRACT

A diagnostic assay system includes a platform configured to receive a disposable cartridge having a sample chamber for receipt of an assay fluid, and an a PCR chamber disposed in fluid communication with the sample chamber for performing target amplification of the assay fluid. A heating source is disposed adjacent a heat exchange surface disposed along at least one side of the PCR chamber and is configured to conform to the contour of the heat exchange surface to accelerate target amplification of the assay fluid. The heating source introduces heat into the assay fluid from one side of the disposable cartridge and, in one embodiment, employs a conformal material interposing the heating source and the heat exchange surface to mitigate the formation of air pockets therebetween.

### PRIORITY CLAIM

This application is a Continuation-In-Part of U.S. patent application Ser. No. 16/303,441 entitled “System and Method for Optimizing Heat Transfer for Target Amplification within a Diagnostic Assay System” which claims priority to U.S. Provisional Patent Application Ser. No. 62/344,711, filed Jun. 2, 2016 entitled “Multi-chamber Rotating Valve and Thermal Control In A Microfluidic Chamber”. The contents of the aforementioned applications are hereby incorporated by reference in their entirety. The contents of the aforementioned applications are hereby incorporated by reference in their entirety. The contents of the aforementioned applications are hereby incorporated by reference in their entirety.

### CROSS-REFERENCE TO RELATED APPLICATIONS

This application also relates to International Patent Application No. PCT/US2017/032904, internationally filed May 16, 2017 entitled “Flow Control System for Diagnostic Assay System”, which claims priority to U.S. Provisional Patent Application Ser. No. 62/337,446 filed May 17, 2016 entitled “Multi-Chamber Rotating Valve and Cartridge.” Additionally, this application also relates to U.S. patent application Ser. No. 15/157,584 filed May 18, 2016 entitled “Method and System for Sample Preparation”, which is a continuation of U.S. Non-Provisional patent application Ser. No. 14/056,543, filed Oct. 17, 2013, now U.S. Pat. No. 9,347,086, which claims priority to U.S. Provisional Patent Application Ser. No. 61/715,003, filed Oct. 17, 2012, which is a continuation-in-part of U.S. patent application Ser. No. 12/785,856, filed May 24, 2010, now U.S. Pat. No. 8,663,918, which claims priority to U.S. Provisional Patent Application Ser. No. 61/180,494, filed May 22, 2009, and which is also a continuation-in-part of U.S. patent application Ser. No. 12/754,205, filed Apr. 5, 2010, now U.S. Pat. No. 8,716,006, which claims priority to U.S. Provisional Patent Application Ser. No. 61/158,519, filed Apr. 3, 2009. The contents of the aforementioned applications are hereby incorporated by reference in their entirety.

### FIELD OF THE INVENTION

The present invention relates to systems and methods for accelerating Polymerase Chain (PC) reactions, and more particularly to efficiently and effectively heating a PCR chamber by a heating source disposed along a single side of the chamber.

### BACKGROUND OF THE INVENTION

There is continuing interest to improve testing methodologies, facilitate collection and decrease the time associated with clinical laboratories. Particular testing requires that a sample be disrupted to extract nucleic acid molecules such as DNA or RNA.

The number of diagnostic tests performed annually has increased exponentially in the past decade. The use of molecular diagnostics and gene sequencing in research and medical diagnostics is also rapidly growing. For example, DNA testing has also exploded in view of the growing interest in establishing and tracking the medical history and/or ancestry of a family. Many, if not all of these assays, could benefit from a rapid sample preparation process that is easy to use, requires no operator intervention, is cost effective and is sensitive to a small sample size.

Sample collection and preparation is a major cost component of conducting real-time Polymerase Chain Reaction (PCR), gene sequencing and hybridization testing. In addition to cost, delays can lead to the spread of infectious diseases, where time is a critical component to its containment/abatement. In addition to delaying the test results, such activities divert much-needed skilled resources from the laboratory to the lower-skilled activities associated with proper collection, storage and delivery.

For example, a portable molecular diagnostic system could be operated by minimally trained personnel (such as described in US 2014/0099646 A1) and have value with regard to disease surveillance. However, the adoption of such portable systems can be limited/constrained by current methods of sample collection, which require trained personnel to permit safe and effective handling of blood/food/biological samples for analysis. Other limitations relate to: (i) the ability of injected/withdrawn fluids to properly flow, (ii) manufacturability, (iii) cross-contamination of assay fluids which may influence the veracity of test results, (iv) proper admixture of assay fluids to produce reliable test results, and (v) the ability or inability to introduce catalysts to speed the time of reaction,

A need, therefore, exists for an improved disposable cartridge for use in combination with a portable molecular diagnostic/assay system which facilitates/enables the use of minimally-trained personnel, hands-off operation (once initiated), repeatable/reliable test results across multiple assay samples (e.g., blood, food, other biological samples) and an ability to cost effectively manufacture the disposable cartridge for the diagnostic assay system.

### SUMMARY OF THE INVENTION

The present invention is directed to an system for performing diagnostic testing of an assay fluid. The diagnostic assay system includes a platform configured to receive a disposable cartridge having a sample chamber for receipt of the assay fluid, and an a PCR chamber disposed in fluid communication with the sample chamber for performing target amplification of the assay fluid. A heating source is disposed adjacent a heat exchange surface disposed along at least one side of the PCR chamber and is configured to conform to the contour of the heat exchange surface to accelerate target amplification of the assay fluid. The heating source introduces heat into the assay fluid from one side of the disposable cartridge and, in one embodiment, employs a conformal material interposing the heating source and the heat exchange surface to mitigate the formation of air pockets therebetween.

### BRIEF DESCRIPTION OF THE DRAWINGS

The present invention is disclosed with reference to the accompanying drawings, wherein:

FIG. 1 is a perspective view of a portable diagnostic assay system operative to accept one of a plurality of disposable cartridges configured to test fluid samples of collected blood/food/biological samples.

FIG. 2 is an exploded perspective view of one of the disposable cartridges configured to test a blood/food/biological sample.

FIG. 3 is a top view of the one of the disposable cartridges illustrating a variety of assay chambers including a central assay chamber, one of which contains an assay chemical suitable to breakdown the fluid sample to detect a particular attribute of the tested fluid sample.

FIG. 4 is a bottom view of the disposable cartridge shown in FIG. 3 illustrating a variety of channels operative to move at least a portion of the fluid sample from one chamber to another the purpose of performing multiple operations on the fluid sample.

FIG. 5 is a perspective view of a portable diagnostic assay system and an exploded view of the requisite components necessary for optimizing target amplification including a mounting platform having a mounting plate, a heat source integrated within the mounting plate, a conductive conformal layer disposed over the mounting plate and a multi-axis actuation system operative to apply a threshold contact force/pressure at a mating interface between the conductive conformal and a fluid channel disposed on an underside surface of the disposable cartridge.

FIG. 6 depicts a profile view of the portable diagnostic assay system depicted in FIG. 5 including a schematic view of the cartridge rotor, the mounting platform, heat source, conformal conductive sheet and the multi-axis actuation system.

FIGS. 7 and 8 depict a schematic view of the multi-axis actuation system of the portable diagnostic assay system moving between an open or disengaged position (FIG. 7) and a closed or engaged position (FIG. 8).

FIG. 9 depicts an enlarged view of the actuation plate together with the conformal conductive elastomeric material disposed over the actuation plate.

FIG. 10 is an enlarged bottom view of the disposable cartridge showing the underside surface thereof including a pair of assay channels for target amplification together with a film of polyurethane material disposed over the assay channels.

FIG. 11 depicts an enlarged cross-sectional view taken substantially along lines 11-11 of FIG. 7.

FIG. 12 depicts an enlarged cross-sectional view taken substantially along lines 12-12 of FIG. 8.

FIG. 13 is a schematic view of another embodiment of the invention wherein the PCR and reaction chambers are integrated.

Corresponding reference characters indicate corresponding parts throughout the several views. The examples set out herein illustrate several embodiments of the invention but should not be construed as limiting the scope of the invention in any manner.

### DETAILED DESCRIPTION

A disposable cartridge is described for use in a portable/automated assay system such as that described in commonly-owned, co-pending U.S. patent application Ser. No. 15/157,584 filed May 18, 2016 entitled “Method and System for Sample Preparation” which is hereby included by reference in its entirety. While the principal utility for the disposable cartridge includes DNA testing, the disposable cartridge may be used to detect any of a variety of diseases which may be found in either a blood, food or biological specimen. For example, blood diagnostic cartridges may be dedicated cartridges useful for detecting hepatitis, autoimmune deficiency syndrome (AIDS/HIV), diabetes, leukemia, graves, lupus, multiple myeloma, etc., just naming a small fraction of the various blood borne diseases that the portable/automated assay system may be configured to detect. Food diagnostic cartridges may be used to detect salmonella, e-coli, staphylococcus aureus or dysentery. Blood diagnostic cartridges may be dedicated cartridges useful for detecting insect or animal borne diseases including malaria, encephalitis and the West Nile virus.

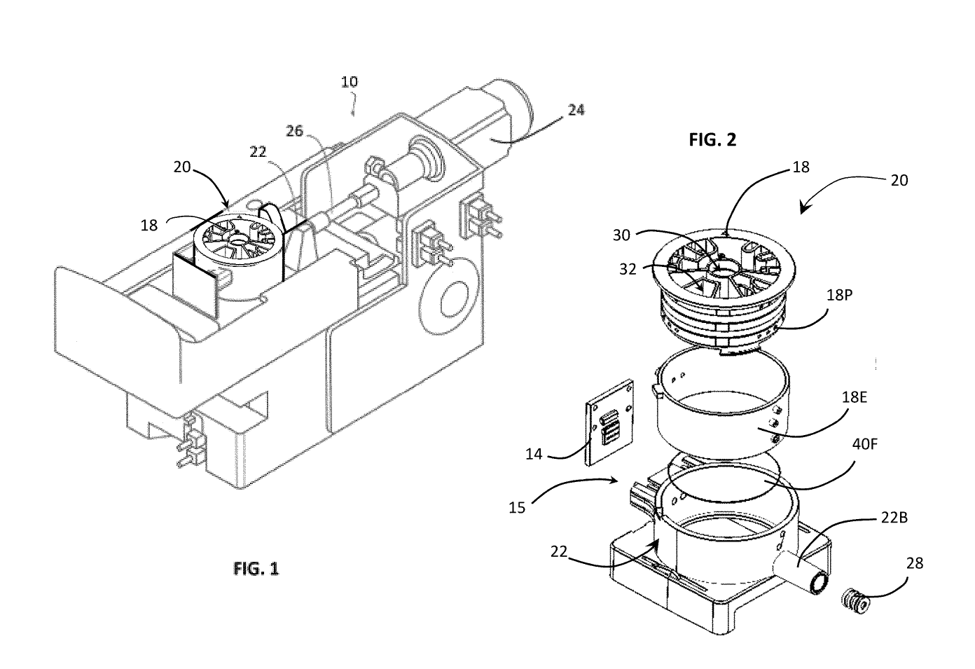

More specifically, and referring to FIGS. 1 and 2, a portable assay system 10 receives any one of a variety of disposable assay cartridges 20, each selectively configured for detecting a particular attribute of a fluid sample, each attribute potentially providing a marker for a blood, food or biological (animal borne) disease. The portable assay system 10 includes one or more linear and rotary actuators operative to move fluids into, and out of, various compartments or chambers of the disposable assay cartridge 20 for the purpose of identifying or detecting a fluid attribute. More specifically, a signal processor 14, i.e., a PC board, controls a rotary actuator (not shown) of the portable assay system 10 so as to align one of a variety of ports 18P, disposed about a cylindrical rotor 18, with a syringe barrel 22B of a stationary cartridge body 22. The processor 14 controls a linear actuator 24, to displace a plunger shaft (not shown) so as to develop pressure, i.e., positive or negative (vacuum) in the syringe barrel 22. That is, the plunger shaft displaces an elastomer plunger 28 within the syringe 22 to move and/or admix fluids contained in one or more of the chambers 30, 32. In addition to controlling the rotary position and plunger apparatus of the disposable cartridge, the processor 14 may be disposed within a reaction chamber 15 and contain the requisite sensors for analysis of the assay fluid. Hence, once the assay fluid has undergone target amplification, e.g., multiplication of the DNA sequence, the fluid may be analyzed in the reaction chamber 15.

The disposable cartridge 20 provides an automated process for preparing the fluid sample for analysis and/or performing the fluid sample analysis. The sample preparation process allows for disruption of cells, sizing of DNA and RNA, and concentration/clean-up of the material for analysis. More specifically, the sample preparation process of the instant disclosure prepares fragments of DNA and RNA in a size range of between about 100 and 10,000 base pairs. The chambers can be used to deliver the reagents necessary for end-repair and kinase treatment. Enzymes may be stored dry and rehydrated in the disposable cartridge, or added to the disposable cartridge, just prior to use. The implementation of a rotary actuator allows for a single plunger to draw and dispense fluid samples without the need for a complex system of valves to open and close at various times. This greatly reduces potential for leaks and failure of the device compared to conventional systems. It will also be appreciated that the system greatly diminishes the potential for human error.

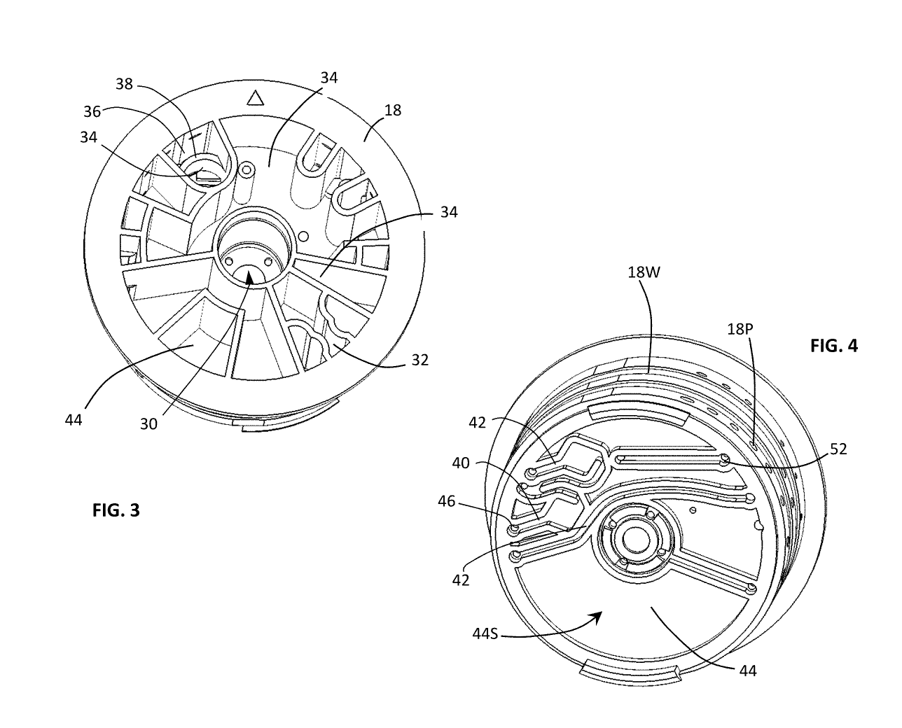

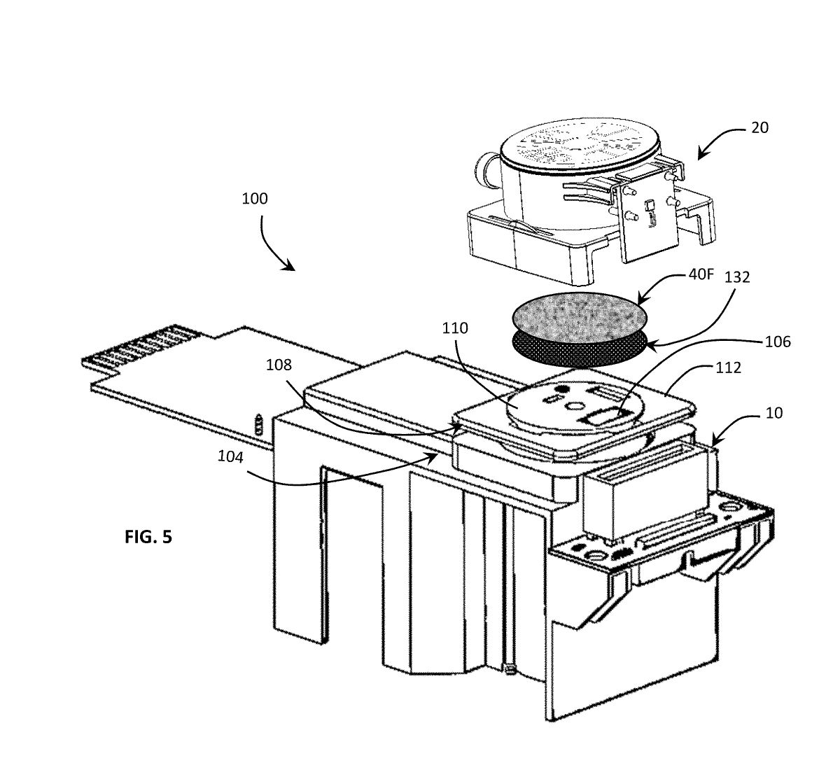

In FIGS. 3 and 4, the cylindrical rotor 18 includes a central chamber 30 and a plurality of assay chambers 32, 34 surrounded, and separated by, one or more radial or circumferential walls. In the described embodiment, the central chamber 30 receives the fluid sample while the surrounding chambers 32, 34 may contain a premeasured assay chemical or reagent for the purpose of detecting an attribute of the fluid sample. The chemical or reagents may be initially dry and rehydrated immediately prior to conducting a test. Some of the chambers 32, 34 may be open to allow the introduction of an assay chemical while an assay procedure is underway or in process. The chambers 30, 32, 34 are disposed in fluid communication, e.g., from one of the ports 18P to one of the chambers 30, 32, 34, by channels 40, 42 molded along a bottom panel 44, i.e., along underside surface of the rotor 18.

Depending upon the specific function of the cartridge 20, one important feature of the channels 40, 42 is to facilitate and augment amplification by forming a region which may be heated from the underside of the cartridge 20. During development of the disposable cartridge and diagnostic assay system, the inventors were faced with various challenges associated with accelerating amplification. More specifically, the inventors learned that the use of conventional conductive grease along the mating interface of a channel 42 was inadequate to reach a desired temperature set point, i.e., to transfer heat, within a reasonable time frame. It was at this point that the inventors began conducting a variety of inventive methods and configurations which would lead to a two-fold increase in amplification time. These tests/inventive discoveries are discussed in the subsequent paragraphs.

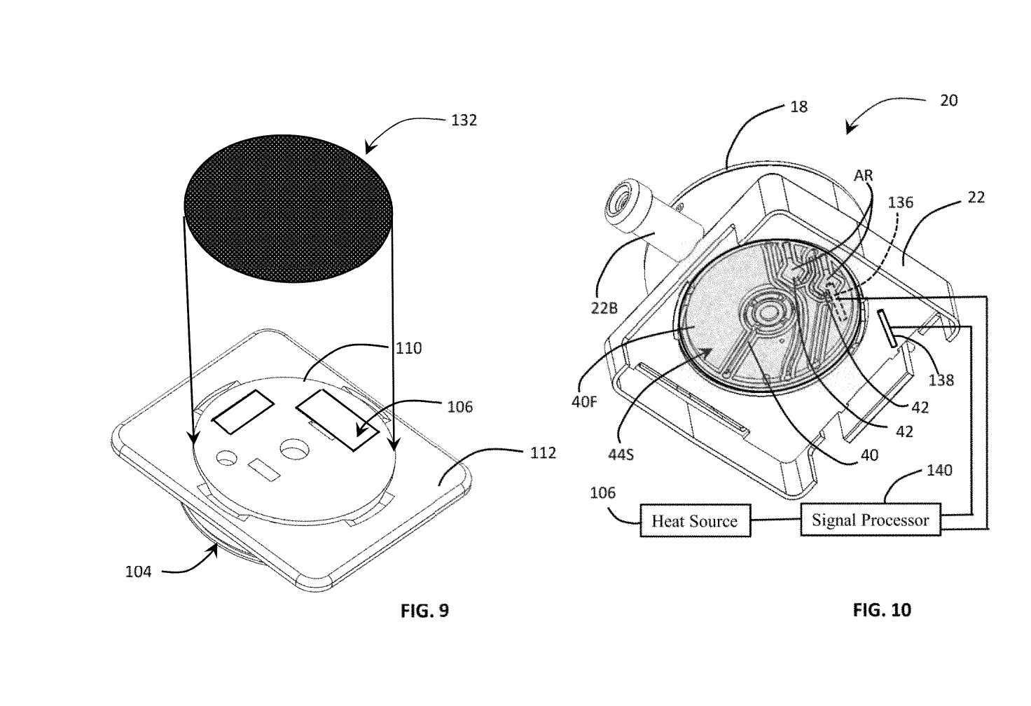

In FIGS. 5, 6, 9 and 10 a diagnostic assay system 100 comprises: (i) a mounting platform 104 configured to receive a disposable cartridge 20; (ii) a heating source or heat source 106 integrated within mounting platform 104, and (iii) an actuation system 108 configured to move a plate 112 of the mounting platform 104 into contact with an underside surface of the disposable cartridge 20. With respect to the latter, the actuation system 108 may rotationally index the rotor 18 of the cartridge 20 into alignment with the syringe barrel of the cartridge body while also displacing the plate 112 into contact with the underside surface of the cartridge 20.

More specifically, the mounting platform 104 includes a circular disc 110 disposed at the center of a rectangular or square mounting plate 112. The circular disc 110 is adjacent to and is contiguous with the underside surface 44S (best seen in FIG. 10) of the disposable cartridge 20. As mentioned in the preceding paragraph, the underside surface 44S of the disposable cartridge 20 forms a network of channels 40, 42, at least one of which facilitates target amplification by providing a PCR chamber AR (FIG. 10) which enhances heat transfer. Specifically, at least one of the channels 42 opens-up or diverges to form a PCR chamber or accumulation region AR where target amplification can occur by rapidly heating the region to a desired or threshold temperature. The PCR chamber or amplification region AR defines a heat transfer/exchange surface or interface which may be covered by a thin film 44F of plastic, however, any suitably thin, low resistivity material will suffice to provide a mating interface for heat transfer, i.e., between the amplification region AR and the circular disc 110.

In the described embodiment, the heat source 106 is integrated with the circular disc 106 of the mounting platform 104. The heat source 106 may be any resistive heater, however, in the disclosed embodiment, a low wattage RF heat source or inductive heater may be employed. That is, inasmuch as the diagnostic assay tester 10 is portable, a source of high current may not be readily available. In view of these contingencies, an RF and/or inductive heater may be preferable inasmuch as such heat sources may operate on 6-12 volt battery power. A typical RF heating device may include any strip of material which is responsive to RF energy. Such materials include a molecular lattice which is excited, i.e., vibrates, in the presence of an RF energy field within a particular frequency band.

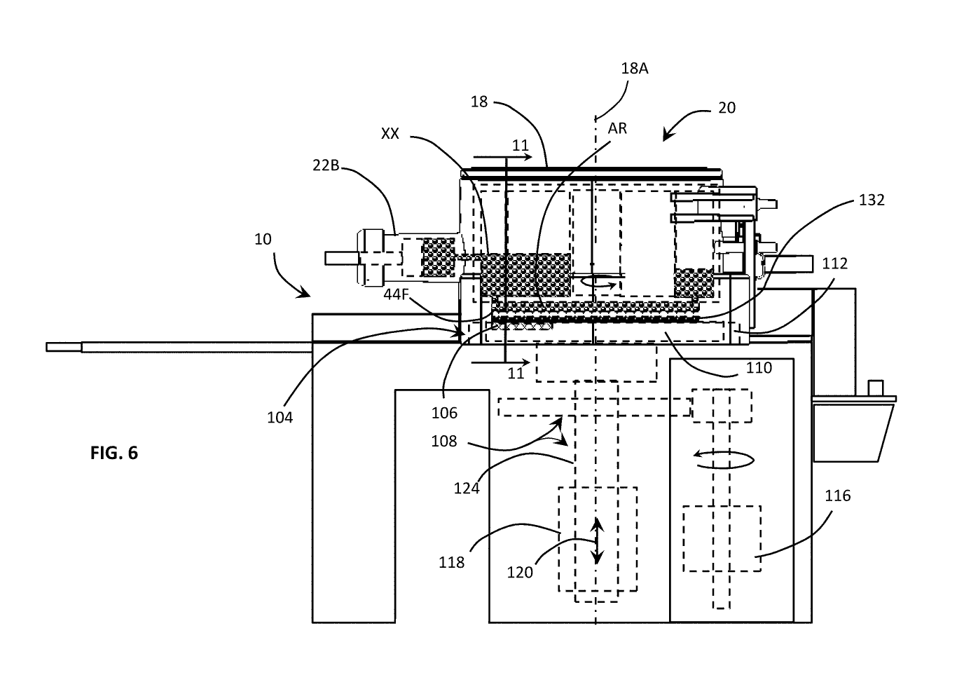

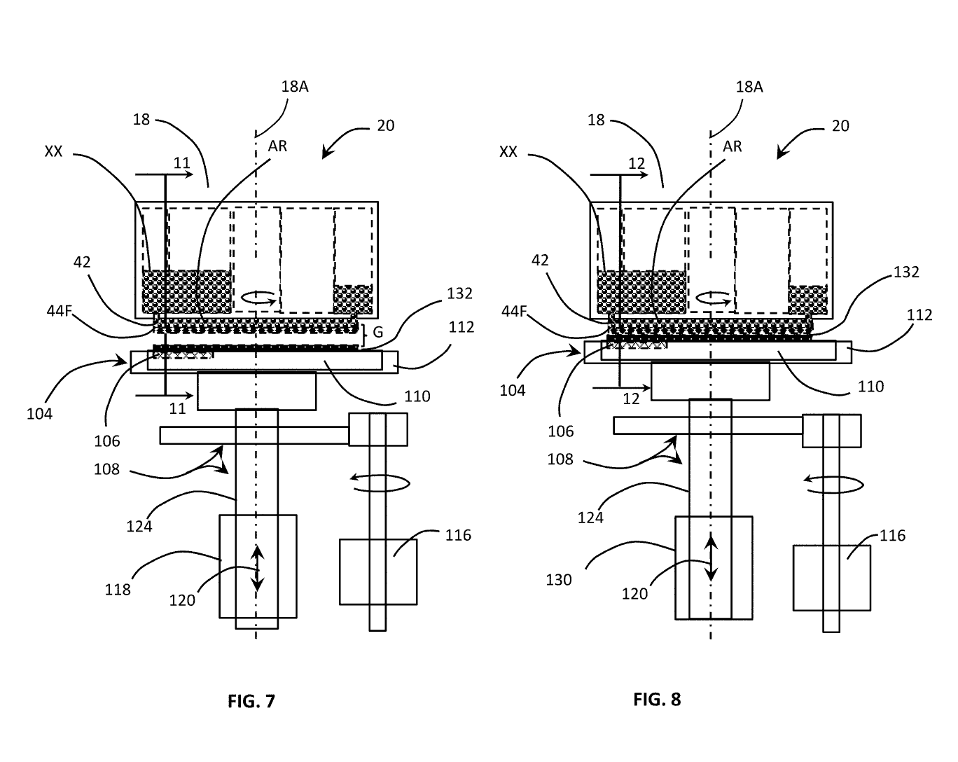

In FIGS. 6, 7 and 8, the multi-axis actuation system 108 integrates with the mounting platform 104 and comprises: (i) a rotary actuator 116 for rotationally indexing the cartridge rotor 18 of the disposable cartridge 20, and a linear actuator 118 operative to apply a contact force/pressure parallel to the rotational axis 18A of the cartridge rotor. While the rotary actuator 116 is shown driving the rotor 18 by pinion/spur gear combination along an axis parallel to the rotational axis 18A of the rotor 18, it will be appreciated that other drive systems are contemplated. For example, greater accuracy and control may be provided by a worm gear (not shown) having an axis perpendicular to the rotational axis 18A. The linear actuator 118 drives a shaft 124 along the rotational axis 18A to induce a contact pressure along a mating interface between the underside surface 44S of the cartridge rotor 18 and the mounting plate 112. FIG. 7 shows the multi-axis actuation system 108 in an open or unengaged position such that the underside surface 44S of the cartridge rotor 18, or the amplification channel 42, is separated from the mounting plate 112 by a gap G. FIG. 8 depicts the multi-axis actuation system 108 in a closed or engaged position such that the mounting plate 112 moves upwardly toward the underside surface 44S of the cartridge rotor 18 until the mounting plate 112, along with the integrated heater 106, is pressed against the fluid channel 42.

In FIGS. 5, 6, and 9-12, the inventors discovered that a number of factors dramatically increased the efficiency and time for target amplification of the sample fluid. In one embodiment, the inventors discovered that by imposing a small contact pressure along the mating interface between the PCR chamber of the amplification channel 42 and the heat source 106, the cycle time required for target amplification was significantly reduced. Additionally, and in another embodiment, it was determined that the addition of a conformal material 130, integrated with, or formed in combination with, the heat source 106, dramatically improves the heat transfer across the heat transfer/exchange surface. It is believed that the combination of an imposed contact force (via an actuated heat source 106) along with a conformal material 132 functions to mitigate the formation of small pockets of air along the mating interface, i.e., air pockets caused by surface roughness along the mating interface. In the described embodiment, the conformal material 132 is between one hundred (100) to five hundred (500) microns in thickness, and more particularly, between one hundred (100) to two hundred (200) microns in thickness.

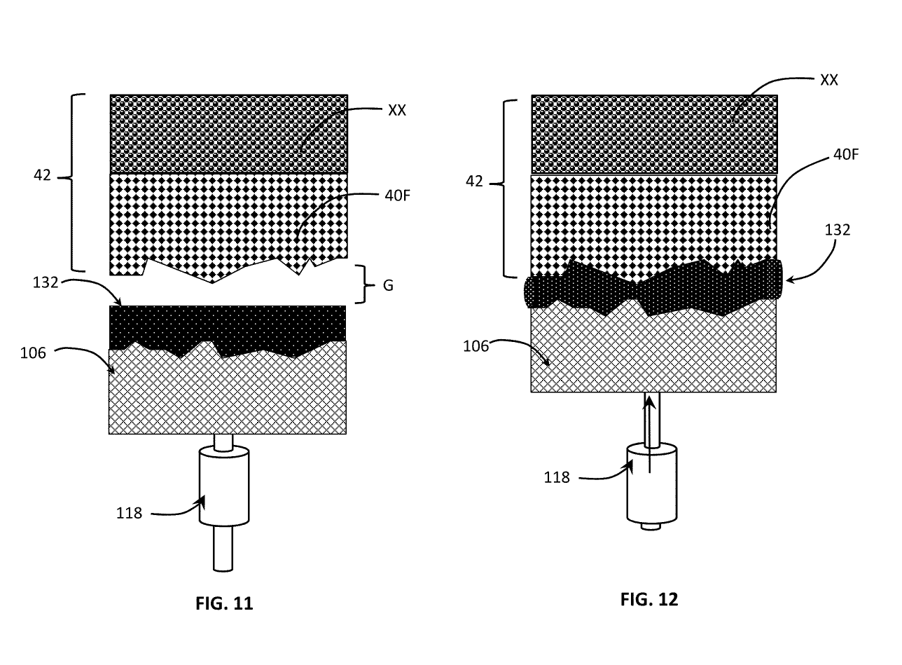

FIG. 11 depicts an enlarged view of the heat source 106, the PC chamber AR or PC channel 42 (comprising the thin film layer 40F which covers the fluid XX), and the conformal layer 132 disposed therebetween. FIG. 12 depicts the same components as those depicted in FIG. 11, but for the linear actuator 118 closing the gap G and imposing a threshold contact pressure along the mating interface. There, it will be appreciated that the small pockets of air generated by the irregular surface of the mating interface, are filled by the conformal layer. As such, heat flows unabated by the insulating pockets of air. In the described embodiment, the threshold contact pressure may be within a range of between about 0.25 lbs./in.² to about 7 lbs./in². More preferably, the threshold contact pressure may be within a range of between about 0.25 lbs./in.² to about 3 lbs./in². Conformal materials which may be used include silicones, elastomers, rubbers, urethanes and films having a low Young's modulus, a high percent elongation (i.e., high strain properties) and/or a low durometer.

With respect to the former, the conformal material 132 is configured to elongate from between about twenty (20%) to about fifty percent (50%) of an original dimension. For example, a conformal material having an original dimension of about 0.5 inches may deform elastically under a tensile load (i.e., pulling the material apart) to between about 0.6 inches to about 0.75 inches. With respect to the latter, it will be appreciated that a conformal material having a Shore-A hardness of between about thirty (30) to about seventy (70) less than about 70 is useful for practicing the inventive features of this disclosure.

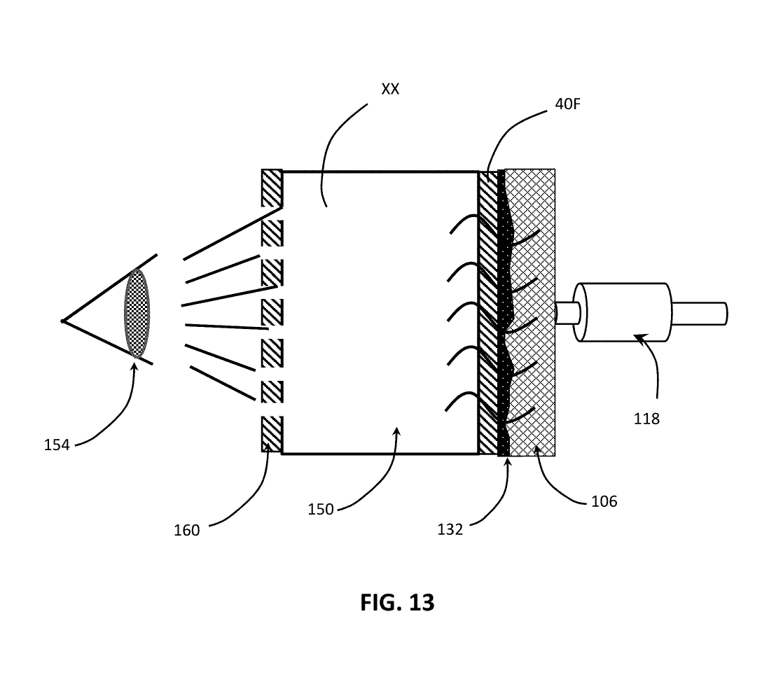

FIG. 13 depicts another embodiment of the disclosure wherein the PC and reaction chambers are integrated or combined such that target amplification and assay fluid analysis are performed in a common chamber 150. In this embodiment, the assay fluid XX is heated along at least one side of chamber while optical or electronic analysis may be performed along another side, i.e., a diametrically opposite side, of the chamber. FIG. 13 shows the fluid XX being heated along one side, i.e., along a side having a conformal coating/material 132 interposed between the heat source 106 and the film 40F, and optically analyzed by a suitable optical device 154 along a slotted side 160 of the reaction chamber 150.

Testing of the various configurations described herein provides nearly a two-fold increase in temperature response and accuracy. For most of the assay fluid procedures, temperatures can be controlled to within one degree Celsius (1°). In one embodiment, a thermocouple 136 may be introduced to measure the temperature within the amplification region AR while another thermocouple 138 reads an ambient temperature to establish a baseline or threshold temperature. The thermocouple 136 in the amplification region AR issues an actual temperature signal indicative of an instantaneous temperature of the assay fluid XX. The signal processor 140 is responsive to the actual temperature signal, compares it to a stored threshold temperature signal, and controls the heat source such that the actual temperature is maintained within a threshold range of the threshold temperature. Alternatively, a second thermocouple 138 issues a baseline or ambient temperature signal for comparison to the actual temperature signal. While the illustrated embodiment depicts a thermocouple along the underside surface of the disposable cartridge 20, it will be appreciated that one or both of the thermocouples 136, 138 may be disposed in combination with the contact plate 112, proximal the heat source 106 and juxtaposed the underside of the cartridge rotor 18.

In one embodiment, the conformal coating is disposed over the heating source. This could be some type of elastomer, silicone, foam, epoxy, phase change material, or gel pack. The properties of the material would be such that repeated contact would have minimal effect on its physical integrity (slow wear). This could be done using slip coatings, slip additives or other fillers. Naturally, the conformal properties would be retained. Alternately, the material may be considered a consumable and replaced after a particular lifetime. This would relax the wear tolerance. While the material would not require thermal conductive properties, it is desired. Materials with a low thermal conductivity would likely require thinner coatings to reduce heat transfer times. With the heating element coated with a conformal and thermally conductive film, the reagent vessel can be put in contact. The conformal nature of the film improves surface to surface contact while minimizing small voids that may occur The temperature can then be cycled.

In another embodiment, the wear and damage incurred are abated by actuating the heating element itself. When not being used, the heating element or heat source is retracted away from the heat transfer surface preventing any contact induced damage. When the process calls for heating or cooling, the heating element or vessel is actuated and the two surfaces are pressed together. The conformal and conductive nature of the heating element surface allows for enhanced surface contact and thermal transfer between the heating element and the reagent vessel. The process of actuating the parts can be done a variety of ways depending upon design requirements.

In another embodiment, the heating element is coated with a conformal and preferably thermally conductive coating. In addition, the heating element is spring loaded. This provided partial wear relief if the vessel and heating element are moved while in contact.

In another embodiment, a material having a high coefficient of thermal expansion is employed while also having a conformal characteristic. The material is coated over the heating element. Under non-processing conditions, the coating would not be in contact with the PC chamber or heat transfer interface. Upon heating, the material expands to fill any voids which may exist between the two surfaces. The enhanced surface contact would allow improved thermal transfer. When processing is complete, the material cools and retracts from the vessel surface to allow free movement therebetween.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

What is claimed is:

1. A diagnostic assay system, comprising:
a platform configured to receive a disposable cartridge having at least one chamber for performing target amplification of an assay fluid, the chamber defining a heat exchange surface; and
a heat source having a compliant heating element configured to: (i) engage the heat exchange surface along one side of the chamber and (ii) conform to the contour of the heat exchange surface to accelerate target amplification of the assay fluid.

2. The diagnostic assay system of claim 1 wherein the disposable cartridge further comprises a sample chamber defining an opening at one end for receipt of the assay fluid, and an a PCR chamber disposed in fluid communication with the sample chamber, the heat exchange surface being disposed along at least one side of the PCR chamber.

3. The diagnostic assay system of claim 1, wherein the disposable cartridge further comprises a reaction chamber, disposed in fluid communication with the PCR chamber, for performing analysis of the assay fluid.

4. The diagnostic assay system of claim 1, wherein the PCR chamber is configured to perform one of an optical and an electronic analysis of the assay fluid.

5. The diagnostic assay system of claim 3, wherein the PCR chamber interposes the sample and reaction chambers.

6. The diagnostic assay system of claim 3, wherein the PCR and reaction chambers are integral.

7. The diagnostic assay system of claim 1, wherein the compliant heating element includes a resistance heater and a conformal material disposed over the resistance heater.

8. The diagnostic assay system of claim 1, wherein the compliant heating element includes a conformal material having conductive particulate suspended therein and wherein the heat source includes an Radio Frequency (RF) energy source.

9. The diagnostic assay system of claim 1, wherein the conformal material is a material from the group of elastomers, silicones, rubbers, urethanes, polyurethanes, and polypropylenes.

10. The diagnostic assay system of claim 7 wherein the conformal material is configured to elongate from between about twenty (20%) to about fifty percent (50%) of an original dimension.

11. The diagnostic assay system of claim 7 wherein the conformal material has a Shore A hardness of between about thirty (30) to about seventy (70).

12. The diagnostic assay system of claim 1 further comprising an actuator disposed in combination with the platform and operative to impose a contact force normal to a plane defined by the heat exchange surface.

13. A diagnostic assay system, comprising:
a disposable cartridge having at least one chamber for performing target amplification of an assay fluid, the at least one chamber defining a heat exchange surface wherein at least a portion thereof comprises a conformal material disposed over a single side of the heat exchange surface;
a platform configured to receive the disposable cartridge; and,
a heat source disposed adjacent the heat exchange surface, the heat source configured to: (i) engage the heat exchange surface and (ii) accelerate target amplification of the assay fluid.

14. The diagnostic assay system of claim 13 wherein the platform further comprises a sample chamber defining an opening at one end for receipt of the assay fluid, and a Polymerase Chain Reaction (PCR) chamber disposed in fluid communication with the sample chamber, the heat exchange surface being disposed along the single side of the PCR chamber.

15. The diagnostic assay system of claim 13, wherein the platform further comprising a Polymerase Chain Reaction (PCR) chamber, disposed in fluid communication with the PCR chamber, for performing analysis of the assay fluid.

16. The diagnostic assay system of claim 14, wherein the PCR chamber is configured to perform one of an optical and an electronic analysis of the assay fluid.

17. The diagnostic assay system of claim 15, wherein the PCR chamber interposes the sample and reaction chambers.

18. The diagnostic assay system of claim 13, wherein the PCR and reaction chambers are integral.

19. The diagnostic assay system of claim 13, wherein the conformal material is a material from the group of elastomers, silicones, rubbers, urethanes, polyurethanes, and polypropylenes.

20. The diagnostic assay system of claim 13 wherein the conformal material is configured to elongate from between about twenty (20%) to about fifty percent (50%) of an original dimension.

21. The diagnostic assay system of claim 13 wherein the conformal material has a Shore A hardness of between about thirty (30) to about seventy (70).

22. The diagnostic assay system of claim 13 wherein the conformal material has a thickness dimension between about one-hundred (100) microns to about two-hundred (200) microns.

23. The diagnostic assay system of claim 13 further comprising an actuator disposed in combination with the platform and operative to impose a contact force normal to a plane defined by the heat exchange surface.

24. A disposable cartridge for analyzing an assay fluid, comprising:
a cartridge body defining a chamber for receipt of the assay fluid; and
a conformal material disposed over one side of the chamber and defining a heat exchange surface;
wherein the cartridge body and the conformal material are disposed adjacent a heat source, and
wherein a mating interface between the conformal material and the heat source mitigate pockets of air from developing across the mating interface to improve the efficacy of heat transfer across the mating interface.

25. The disposable cartridge of claim 24 wherein the conformal material is a compliant material loaded with a conductive particulate.

26. The disposable cartridge of claim 24 wherein the conformal material is a material from the group of elastomers, silicones, rubbers, urethanes, polyurethanes, and polypropylenes.

27. The disposable cartridge of claim 24 wherein the conformal material has elongation properties between about twenty percent (20%) to about fifty percent (50%).

28. The diagnostic cartridge of claim 24 wherein the conformal material has durometer of about a Shore A hardness of between about thirty percent (30%) to about seventy percent (70%).

29. The diagnostic assay cartridge of claim 24, wherein the conformal material has a thickness dimension between about one-hundred (100) microns to about five-hundred (500) microns.

30. The diagnostic assay cartridge of claim 24 wherein the conformal material has a thickness dimension between about one-hundred (100) microns to about two-hundred (200) microns.

31. A method for target amplification of an assay fluid, comprising the steps of:
loading a disposable cartridge into a diagnostic assay system, the disposable cartridge having at least one chamber for receiving the assay fluid and performing target amplification of the assay fluid, the chamber defining at least one heat exchange surface along a side thereof;
providing a heat source proximal to the diagnostic assay system; and
producing a conformal mating interface between the heat source and the at least one heat exchange surface to mitigate pockets of air from developing across the conformal mating interface.

32. The method of claim 31, wherein the method further comprises the step of:
configuring the heat source such that a surface which forms a portion of the conformal mating interface is fabricated from a conformal material.

33. The method of claim 32, wherein the method further comprises the step of:
configuring the at least one heat exchange surface which forms a portion of the conformal mating interface is fabricated from a conformal material.

34. The method of claim 32, wherein the step of configuring the at least one heat exchange surface comprises:
loading the conformal material with a conductive particulate.

35. The method of claim 33, wherein the step of configuring the at least one heat exchange surface comprises:
fabricating the at least one heat exchange surface from the group including elastomers, silicones, rubbers, urethanes, polyurethanes, and polypropylenes.

36. The method of claim 32, wherein the conformal material has elongation properties between about twenty percent (20%) to about fifty percent (50%).

37. The method of claim 32, wherein the conformal material has durometer of about a Shore A hardness of between about thirty percent (30%) to about seventy percent (70%).

38. The method of claim 32, wherein the conformal material has a thickness dimension between about one-hundred (100) microns to about five-hundred (500) microns.

39. The method of claim 32, wherein the conformal material has a thickness dimension between about one-hundred (100) microns to about two-hundred (200) microns.
